(19) United States
(12) Patent Application Publication
(10) Pub. No.: US 2026/0166038 A1
(43) Pub. Date: Jun. 18, 2026

(54) METHOD OF ENHANCING THE EFFICACY OF A TARGETED PROTEIN DEGRADER AND ITS DERIVATIVE THERAPEUTICS

(71) Applicant: ShanghaiTech University, Shanghai (CN)

(72) Inventors: Yang LIU, Shanghai (CN); Tianyu SONG, Shanghai (CN); Bo PENG, Shanghai (CN); Yong CANG, Shanghai (CN)

(73) Assignee: ShanghaiTech University, Shanghai (CN)

(21) Appl. No.: 18/863,345

(22) PCT Filed: Apr. 21, 2023
(86) PCT No.: PCT/CN2023/089697

§ 371 Date: Aug. 26, 2025

(30) Foreign Application Priority Data
May 6, 2022 (CN) 202210537433.5

(51) Int. Cl. A61K 31/519; A61K 31/404; A61K 31/436; A61K 31/454; A61K 31/496; A61K 47/55; A61P 35/00

(52) U.S. Cl. CPC A61K 31/519; A61K 31/404; A61K 31/436; A61K 31/454; A61K 31/496; A61K 47/55; A61P 35/00

(57) ABSTRACT

A method of enhancing the efficacy of a targeted protein degrader and its derivative therapeutics is provided. The mTOR inhibitor can significantly enhance the degradation of substrates by targeted protein degraders such as molecular glue degraders and PROTACs, thereby achieving therapeutic purposes. Moreover, the combination therapy is particularly suitable for myeloma patients who have developed resistance to immunomodulatory drugs (IMiDs), a kind of molecular glue degrader, of which the continuous use resulting in reduced treatment efficacy and myeloma relapse. Also provided is a pharmaceutical composition, which comprises a targeted protein degrader and its derivative therapeutics, and a mTOR inhibitor. By promoting the degradation of substrates by targeted protein degrader and its derivative therapeutics, the effectiveness of such drugs in treating disease can be increased.

### FIELD

The present disclosure relates to the technical field of biomedicine and discloses the application of mTOR inhibitors in enhancing the efficacy of targeted protein degrader and its derivative therapeutics.

### BACKGROUND

Immunomodulatory drugs (IMiDs) primarily include thalidomide and its derivatives (lenalidomide, pomalidomide, CC-122 and CC-220), and are classified as molecular glue degraders (MGD). In the 1990s, a series of studies demonstrated that thalidomide exhibited significant anti-myeloma efficacy, re-establishing its prominence in the clinic. To alleviate the teratogenic effects of thalidomide on fetuses, lenalidomide, pomalidomide, and other derivatives were synthesized based on thalidomide's structure. Thalidomide and its derivatives are referred to as immunomodulatory drugs due to their regulatory impact on the immune system. Immunomodulatory drugs can stimulate the proliferation of T-cells maintained in interleukin-2 (IL-2) by augmenting the secretion of interferon-gamma (IFN-γ) and can inhibit tumor formation by suppressing TNF-α and IL-1β. Research indicates that these medications not only markedly reduce symptoms in early-stage myeloma patients but also help treat patients with refractory and relapsed myeloma, for which they are regarded as premier pharmaceuticals and the primary therapies for myeloma. Nevertheless, these medications cannot entirely eradicate myeloma and exhibit diminished efficacy in addressing relapsed myeloma. Those patients with relapsed myeloma possess limited effective treatment alternatives.

In 2014, Ben Ebert's team from Harvard Medical School and William Kealin Jr's team, a Nobel Prize laureate in Physiology or Medicine, simultaneously published their independent research on Science, demonstrating the molecular mechanism by which immunomodulatory drugs (IMiDs) treat myeloma. They demonstrated that IMiDs can bind to Cereblon (CRBN) and hijack the CRL4 (CRBN) E3 ubiquitin ligase to recognize new substrates—two transcription factors, IKZF1 and IKZF3. Since IKZF1 and IKZF3 play a critical role in the growth and proliferation of myeloma cells, IMiDs have a cytotoxic effect on tumor cells. Researches on Nature and its sub-journals provided structural confirmation of this mechanism later. The ubiquitination pathway utilized by IMiDs is a critical physiological process that controls post-translational modification. The body maintains homeostasis by covalently attaching a 76-amino acid protein known as ubiquitin to target proteins. Typically, ubiquitin-activating enzymes (E1) activate and transfer ubiquitin to ubiquitin-conjugating enzymes (E2), while ubiquitin ligases (E3 ligases) bind to target proteins and transfer ubiquitin from E2 to the target proteins. Finally, the 26S proteasome recognizes the polyubiquitin chain and degrades the target proteins. In this process, E3 ubiquitin ligase plays an important role in substrate recruitment.

The immunomodulatory drugs known as IMiDs are the first examples of small compounds that have been found to specifically target and hijack E3 ubiquitin ligases for the degradation of novel substrates. IMiDs attach to Cereblon (CRBN) to form a new surface that facilitates the ubiquitination and destruction of IKZFs. Traditional inhibition works by using chemicals to bind pockets and disrupt enzyme activities. However, most pathogenic proteins, including oncogenic proteins, lack pocket structures. The clarification of the molecular mechanism of IMiDs has demonstrated to scientists the ability to target and destroy formerly “undruggable” proteins. In 2015, Bradner's team at Harvard Medical School published on Science the successful conjugation of the potent acute myeloid leukemia (AML) agent JQ-1 with a phthalimide framework that exhibits strong affinity for CRBN. The compound, designated as dBET1 in the study, can swiftly and thoroughly degrade BRD4, thereby suppressing the expression of essential growth genes for tumor proliferation. This initiated a surge in the R&D procedure of proteolysis-targeting chimeras (PROTAC) pharmaceuticals. It became clear that conventional protein inhibitors could only repress the activity of one domain of the target protein. However, cancer-associated proteins generally execute many activities through multiple domains. Therefore, traditional drugs often result in activation of compensatory genes or pathways in organisms, which then leads to drug resistance. The targeted ubiquitination and destruction of associated proteins can mitigate such problems, which in terms facilitate the advancement of PROTAC. By linking the target protein at one end and the ubiquitination degradation system at the other, PROTACs can persistently and completely destroy target proteins, diminishing the necessary drug concentration while providing excellent selectivity and rapidity. A number of PROTACs have been designed, with numerous candidates progressing towards clinical trials. Furthermore, PROTACs that either target hitherto challenging or undruggable targets have also been developed, including KRAS degraders.

However, PROTACs have poor absorption and safety issues due to their high molecular weight. MGDs, which are exemplified by IMiDs or degraders developed based on IMiDs' structure, offer the benefits of PROTACs in terms of target protein degradation while avoiding the disadvantages of large molecular weight. The creation of PROTACs and MGDs using the knowledge and designs gleaned from IMiDs is showing a promising result as a novel approach to the manufacturing of anti-tumor medications.

The mTOR signaling pathway plays a critical function in cell survival and metabolism by facilitating the exchange of chemicals inside and outside the cells. From cellular autophagy to protein synthesis, the mTOR signaling system is implicated in many essential life functions. The mTOR signaling pathway depends on two major complexes: mTORC1 and mTORC2. To promote pyrimidine synthesis, mTORC1 can phosphorylate the downstream substrate S6K1, which in turn phosphorylates carbamoyl-phosphate synthetase (CAD). By phosphorylating the substrate 4EBP and forcing it to separate from eIF4E, mTORC1 can further stimulate mRNA translation. Numerous studies have also indicated that resistance to various anti-tumor medications is probably regulated by the mTOR signaling pathway. The mTOR signaling system can be inhibited by rapamycin and its derivatives, including everolimus. Everolimus has been licensed for the treatment of melanoma, colorectal cancer, and other conditions. It has better water solubility than rapamycin and suppresses the proliferation of vascular endothelial cells. As of right now, there are no reports of mTOR inhibitors improving the effectiveness of therapies using targeted protein degraders and their derivatives.

### SUMMARY

The present disclosure provides a method of enhancing the efficacy of a targeted protein degrader and its derivative therapeutics. The present disclosure enhances the ability of first-line clinical targeted protein degrader and its derivative therapeutics, such as immunomodulatory drugs (IMiDs), to degrade substrates and improve the efficacy of targeted protein degrader and its derivative therapeutics. Through whole-genome CRISPR screening, mTOR signaling pathway inhibitors, such as rapamycin and everolimus, were found to be capable of enhancing the degradation of substrates by molecular glue degraders (MGDs) and Proteolysis Targeting Chimeras (PROTACs). The combined use of mTOR signaling pathway inhibitors is expected to improve the therapeutic effects of targeted protein degraders represented by IMiDs in clinical treatments.

The present disclosure provides the application of mTOR inhibitors in the preparation of products for enhancing the efficacy of targeted protein degrader and its derivative therapeutics.

The present disclosure also provides a pharmaceutical composition, which comprises a targeted protein degrader and its derivative therapeutics, a mTOR inhibitor, and pharmaceutically acceptable excipients, wherein the targeted protein degrader and its derivative therapeutics comprise a MGD and/or a PROTAC.

The beneficial effect of the present disclosure is as follows:

The mTOR inhibitors proposed in the present disclosure can significantly enhance the degradation of substrates by targeted protein degraders, such as MGDs and PROTACs, thereby achieving therapeutic purposes. This is particularly beneficial for myeloma patients who have developed resistance to immunomodulatory drugs (IMiDs), a type of molecular glue degrader, leading to decreased treatment efficacy and disease relapse. The combined use of mTOR inhibitors can help treat such resistant or relapsed patients, improving the therapeutic effects of existing clinical drugs. Various new-generation targeted protein degraders and their derivative therapeutics, including MGDs and PROTACs targeting substrates besides IKZFs, are under development and even in clinical trials, guiding the new design approaches of drugs beyond the traditional small molecule inhibitors. The combination with mTOR inhibitors enhances their efficacy further and has great therapeutic potential.

### BRIEF DESCRIPTION OF THE DRAWINGS

FIGS. 1A-1E show that activation of the mTOR signaling pathway can confer resistance to immunomodulatory drugs in OPM2 myeloma cells. FIG. 1A represents construction of DEPDC5 knockout cell lines in OPM2 myeloma cells; FIGS. 1B-E show killing curves of immunomodulatory drugs pomalidomide (B), lenalidomide (C), CC-220 (D), and CC-122 (E) on DEPDC5 knockout OPM2 cells.

FIGS. 2A-2E show the combination of pomalidomide and the mTOR signaling pathway inhibitor rapamycin has a strong cytotoxic effect on myeloma cells. FIGS. 2A-E show killing curves of pomalidomide (Poma), rapamycin (Rapa), and their combination on myeloma cell lines MM1S (A), H929 (B), OPM2 (C), U266 (D), and RPMI-8226 (E).

FIGS. 3A-3C show that rapamycin enhances the degradation of IKZF3 and IKZF1 by pomalidomide. FIGS. 3A-C represent protein expression levels of substrates like IKZF3 in myeloma cell lines OPM2 (A), RPMI-8226 (B), and U266 (C), following treatment with pomalidomide, rapamycin, and their combination, as detected by Western blot.

FIGS. 4A-B show that rapamycin enhances the degradation of GSPT1 by CC90009 and CC-885. FIGS. 4A-B represent protein expression levels of substrate GSPT1 in HL60 (A) and MOLT-4 (B) cells, following treatment with CC90009, rapamycin, and their combination, as detected by Western blot.

FIGS. 5A-C show that rapamycin enhances the degradation of substrates by sulfonamides and PROTACs. FIGS. 5A-C represent protein expression levels of substrates in OPM2 cells, following treatment with sulfonamide such as indisulam (A) and PROTACs such as ZNL-02-096 (B) and ARV-771 (C) alone, as well as in combination with rapamycin, as detected by Western blot.

FIGS. 6A-6B show that everolimus, a mTOR signaling pathway inhibitor, enhances the degradation of IKZF3 and IKZF1 by pomalidomide. FIG. 6A represents protein expression levels of substrate IKZF3 in OPM2 cells treated with pomalidomide, everolimus, and their combination, as detected by Western blot. FIG. 6B represents protein expression levels of substrate GSPT1 in KP4 cells treated with CC90009, CC885, and their combinations with rapamycin or everolimus, as detected by Western blot.

FIGS. 7A-7C show that rapamycin enhances the degradation of IKZF3 by CC-92480 and improves the cytotoxicity of CC-92480 on myeloma cells. FIG. 7A represents protein expression levels of substrate IKZF3 in pomalidomide-resistant OPM2-P5000 myeloma cells treated with CC-92480, rapamycin, and their combination, as detected by Western blot. FIG. 7B represents protein expression levels of substrate IKZF3 in pomalidomide-sensitive OPM2 myeloma cells treated with CC-92480, rapamycin, and their combination, as detected by Western blot. FIG. 7C shows killing curves of pomalidomide, CC-92480, rapamycin, and the first two aforementioned drugs in combination with rapamycin on OPM2-P5000 myeloma cells.

### DETAILED DESCRIPTION

Embodiments of the present disclosure are hereinafter described by way of particular specific examples, and other advantages and efficacies of the present disclosure may be readily appreciated by those skilled in the art according to what is disclosed herein. The present disclosure may also be implemented or applied in various other specific embodiments, and various details in this specification may be modified or altered based on different viewpoints and applications without departing from the spirit of the present disclosure.

Experiments in various human myeloma cell models are conducted in the present disclosure. Through whole-genome CRISPR screening in vitro, it is found that the activity of the mTOR signaling pathway plays a crucial role in myeloma cells' resistance to immunomodulatory drugs (IMiDs). The data indicate that combining with mTOR signaling pathway inhibitors can significantly enhance the degradation of substrates by targeted protein degraders and its derivative therapeutics including immunomodulatory drugs and other MGDs (such as CC-90009, CC-885, CC-92480 (Mezigdomide), and sulfonamides), and PROTACs (such as ZNL-02-096 and ARV-771), in both hematologic and solid tumor cell lines, thereby improving their efficacy. This suggests that clinically combining mTOR inhibitors (such as rapamycin and everolimus) can effectively enhance the therapeutic effects of targeted protein degraders and their derivative therapeutics.

The present disclosure provides the use of mTOR inhibitors in the preparation of products for enhancing the efficacy of targeted protein degrader and its derivative therapeutics.

The mTOR inhibitors refer to allosteric inhibitors or catalytic inhibitors capable of inhibiting the mTOR signaling pathway. Preferably, these mTOR inhibitors include one or more of the following: Rapamycin (Sirolimus), Everolimus, Temsirolimus, Ridaforolimus (deforolimus, MK-8669), Sapanisertib (MLN0128), Vistusertib (AZD2014), and CC-115.

In some embodiments, the catalytic inhibitors are kinase inhibitors, e.g., AKT inhibitors.

In some embodiments, the mTOR inhibitors inhibit the activation of mTORC1 and mTORC2.

In some embodiments, the mTOR inhibitors inhibit the activation of one or more of PI3K protein, AKT protein, mTOR protein, S6K1 protein, and 4EBP1 protein in the PI3K/Akt/mTOR signaling pathway.

In some embodiments, the mTOR inhibitors are anti-tumor drugs acting through the mTOR signaling pathway, and/or drugs for treating diabetes, and/or drugs for treating Alzheimer's disease, and/or drugs for delaying aging.

In some embodiments, the mTOR inhibitors are anti-tumor drugs acting through the mTOR signaling pathway and inhibit the activation of at least one or more of PI3K protein, AKT protein, mTOR protein, S6K1 protein, and 4EBP1 protein in the PI3K/Akt/mTOR signaling pathway.

In the present disclosure, the targeted protein degrader and its derivative therapeutics are selected from MGDs and PROTACs. Preferably, the targeted protein degrader and its derivative therapeutics are MGDs; more preferably, the targeted protein degrader and its derivative therapeutics are immunomodulatory drugs.

The MGDs comprise one or more of thalidomide derivatives, CC-90009, CC-885, indisulam, and CC-92480; preferably, the thalidomide derivatives comprise thalidomide, lenalidomide, pomalidomide, CC-122, and CC-220.

The PROTACs comprise one or more of ZNL-02-096, ARV-771, ARV-110, ARV-471, KT-474, and NX-2127.

In the present disclosure, in some embodiments, the mTOR inhibitors are used to enhance the ability of targeted protein degrader and its derivative therapeutics to degrade substrates.

In the present disclosure, in some embodiments, the mTOR inhibitors are used to enhance the sensitivity of substrates to the targeted protein degrader and its derivative therapeutics.

In the present disclosure, in some embodiments, the mTOR inhibitors are used to reduce resistance to the targeted protein degrader and its derivative therapeutics.

In the present disclosure, in some embodiments, the mTOR inhibitors are used to decrease the viability of tumor cells.

In the present disclosure, in some embodiments, the mTOR inhibitors are used to treat diseases targeted by the targeted protein degrader and its derivative therapeutics.

In the present disclosure, in some embodiments, the diseases comprise one or more of tumors, neurological diseases, autoimmune diseases, infectious diseases, and inflammatory diseases. The tumors comprise lymphoma, hematoma, or solid tumors. Preferably, the tumors are one or more of adrenocortical carcinoma, uroepithelial carcinoma of the bladder, breast carcinoma, squamous cell carcinoma of the cervix, intracervical adenocarcinoma, cholangiocarcinoma, adenocarcinoma of the colon, lymphoid neoplasm, diffuse large B-cell lymphoma, esophageal carcinoma, pleomorphic glioblastoma, squamous cell carcinoma of the head and neck, smectochromatous cell carcinoma of the kidney, clear cell carcinoma of the kidney and papillary cell carcinoma of the kidney, acute myeloid leukemia, low-grade glioma of the brain, hepatocellular carcinoma, adenocarcinoma of the lung, squamous cell carcinoma of the lung, mesothelial cell carcinoma, ovarian carcinoma, pancreatic carcinoma, pheochromocytoma and paraganglioma, prostate carcinoma, rectal carcinoma, malignant sarcoma, melanoma, gastric carcinoma, testicular germ-cell tumors, thyroid cancer, thymus gland carcinoma, endometrial carcinoma, uterine sarcoma, uveal melanoma, multiple myeloma, acute gonorrheal leukemia Multiple myeloma, acute lymphoid leukemia, chronic lymphoid leukemia, chronic myelogenous leukemia, T-cell lymphoma and B-cell lymphoma; more preferably, the tumor is one or more of myeloma, leukemia, and pancreatic cancer.

The neurological diseases comprise one or more of Parkinson's disease, Huntington's disease, multiple system atrophy, motor neuron diseases, Alzheimer's disease, traumatic brain injury, ischemic stroke, spinal cord diseases, amyotrophic lateral sclerosis, multiple sclerosis, and seizures.

The autoimmune diseases comprise one or more of systemic lupus erythematosus, rheumatoid arthritis, systemic sclerosis, Sjogren's syndrome, and polymyositis.

The tumor is myeloma; preferably, the myeloma cells include MM1S, NCI-H929, OPM2, U266, and RPMI-8226.

In some embodiments, the rapamycin enhances the killing effect of pomalidomide in myeloma cells; preferably, the myeloma cells comprise at least one of myeloma cell lines MM1S, H929, and OPM2, which are sensitive to the immunomodulatory drug pomalidomide, and at least one of myeloma cell lines U266 and RPMI-8226, which are resistant to pomalidomide.

In some embodiments, the rapamycin enhances the degradation of substrates IKZF3 and IKZF1 by pomalidomide; preferably, the rapamycin enhances the degradation of substrates IKZF3 and IKZF1 by pomalidomide in the myeloma cell lines OPM2, RPMI-8226, or U266.

In some embodiments, the rapamycin enhances the degradation of the substrate GSPT1 by CC-90009 and CC-885; preferably, the rapamycin enhances the degradation of the substrate GSPT1 by CC-90009 and CC-885 in the leukemia cell lines HL60 and MOLT-4.

In some embodiments, the rapamycin enhances the degradation of substrates by the sulfonamide such as indisulam; preferably, the rapamycin enhances the degradation of the substrate RBM39 by the sulfonamide such as indisulam in the myeloma cell line OPM2.

In some embodiments, the rapamycin enhances the degradation of substrates by the PROTACs such as ZNL-02-096 and ARV-771; preferably, the rapamycin enhances the degradation of the substrate Wee1 by the PROTAC ZNL-02-096 and enhances the degradation of the substrate BRD4 by the PROTAC ARV-771.

In some embodiments, the everolimus enhances the degradation of substrates by pomalidomide; preferably, the everolimus enhances the degradation of substrates IKZF3 and IKZF1 by pomalidomide in the myeloma cell line OPM2.

In some embodiments, the rapamycin enhances the degradation of substrates by CC-90009 and CC-885; preferably, the rapamycin enhances the degradation of the substrate GSPT1 by CC-90009 and CC-885 in solid tumor cell lines and the human pancreatic cancer cell line KP4.

In some embodiments, the everolimus enhances the degradation of substrates by CC-90009 and CC-885; preferably, the everolimus enhances the degradation of the substrate GSPT1 by CC-90009 and CC-885 in solid tumor cell lines and human pancreatic cancer cell line KP4.

In some embodiments, the rapamycin enhances the degradation of substrates by CC-92480. In some embodiments, the rapamycin enhances the degradation of the substrate IKZF3 by CC-92480. In some embodiments, the rapamycin enhances the degradation of the substrate IKZF3 by CC-92480 in myeloma cell lines. In some embodiments, the rapamycin enhances the degradation of the substrate IKZF3 by CC-92480 in a pomalidomide-sensitive or pomalidomide-resistant myeloma cell line. In some embodiments, the rapamycin enhances the degradation of the substrate IKZF3 by CC-92480 in the pomalidomide-sensitive myeloma cell line OPM2, as well as in the pomalidomide-resistant myeloma cell line OPM2-P5000.

In some embodiments, the rapamycin enhances the killing effect of CC-92480 on myeloma cells. In some embodiments, the rapamycin enhances the killing effect of CC-92480 on pomalidomide-sensitive or pomalidomide-resistant myeloma cells. In some embodiments, the rapamycin enhances the killing effect of CC-92480 on the pomalidomide-sensitive myeloma cell line OPM2 or the pomalidomide-resistant myeloma cell line OPM2-P5000.

The present disclosure also provides a pharmaceutical composition, which comprises a targeted protein degrader and its derivative therapeutics and a mTOR inhibitor, as well as pharmaceutically acceptable excipients. The pharmaceutical composition described herein may be used to treat one or more of tumors, neurological diseases, autoimmune diseases, infectious diseases, and inflammatory diseases.

In the pharmaceutical composition of the present disclosure, the active components, which include the targeted protein degrader and its derivative therapeutics and the mTOR inhibitor, are typically present in safe and effective amounts. These safe and effective amounts can be adjusted by those skilled in the art, depending on factors such as the patient's body weight, route of administration, and the condition and severity of the disease, e.g., the amounts of the active components to be administered can usually be in a range of 1˜1000 mg/kg/day, 20˜200 mg/kg/day, 1˜3 mg/kg/day, 3˜5 mg/kg/day, 5˜10 mg/kg/day, 10˜25 mg/kg/day, 25˜30 mg/kg/day, 30˜40 mg/kg/day, 40˜60 mg/kg/day, 60˜80 mg/kg/day, 80˜100 mg/kg/day, 100˜150 mg/kg/day, 150˜200 mg/kg/day, 200˜300 mg/kg/day, 300˜500 mg/kg/day, or 500˜1000 mg/kg/day.

One skilled in the art may determine the effective amount to be administered depending on the severity of the disease and the health and age of the subject. The effective amount may typically be in the range of 0.05 ng/kg body weight˜100 mg/kg body weight.

In the present disclosure, the pharmaceutical composition can be adapted for any form of administration, such as oral, nasal, rectal, intravenous, and parenteral administrations. The pharmaceutical composition can be formulated into injections, sterile powders for injection, tablets, pills, capsules, lozenges, elixirs, powders, granules, syrups, solutions, tinctures, aerosols, sprays, or suppositories.

The targeted protein degrader and its derivative therapeutics and mTOR inhibitors in the pharmaceutical composition may be co-administered. “Co-administration” means simultaneous administration by the same or different routes or sequential administration by the same or different routes, either in the same formulation or in two different formulations. “Sequential administration” means there may be a time difference of seconds, minutes, hours, or days between the administration of the targeted protein degrader and its derivative therapeutics and the mTOR inhibitor.

The targeted protein degrader and its derivative therapeutics and the mTOR inhibitor, as well as the pharmaceutical composition in the present disclosure, may be combined with other treatment methods, including surgery, radiotherapy, chemotherapy, and targeted therapy.

The main targets of the targeted protein degrader and its derivative therapeutics and the mTOR inhibitor, as well as the pharmaceutical composition in the present disclosure, are mainly targeted at mammals or their in vitro cancer cells. The preferred mammals are rodents, artiodactyls, perissodactyls, lagomorphs, primates, etc. The primates are preferably monkeys, apes, or humans.

The thalidomide derivatives in the present disclosure comprise pharmaceutically acceptable salts, esters, isomers, prodrugs, polymorphs, and solvates of thalidomide. The pharmaceutically acceptable salts and esters comprise those formed by thalidomide and acids, and the acids are selected from one or more of the following: hydrochloric acid, hydrobromic acid, phosphoric acid, lactic acid, pyruvic acid, acetic acid, succinic acid, oxalic acid, fumaric acid, maleic acid, oxaloacetic acid, methanesulfonic acid, ethanesulfonic acid, sulfuric acid, citric acid, tartaric acid, benzenesulfonic acid, and hydroxyethyl sulfonic acid.

“Prodrug” refers to a compound that, when administered using an appropriate method, will undergo metabolism or chemical reaction in the body to convert itself into the active drug or its pharmaceutically acceptable salt, ester, isomer, prodrug, polymorph, or solvate.

The present disclosure also provides a method for treating diseases in a subject in need, comprising the administration of a therapeutically effective amount of the targeted protein degrader, its derivative therapeutics, and the mTOR inhibitor or the pharmaceutical composition to an individual or subject in need thereof. The diseases are selected from one or more of tumors, neurological diseases, autoimmune diseases, infectious diseases, and inflammatory diseases.

The term “treat” as used herein refers to administering one or more of the targeted protein degrader and its derivative therapeutics, the mTOR inhibitors, and the pharmaceutical compositions described above to a subject, e.g., a mammal (such as a human) suffering from the described diseases or having symptoms of the described diseases, to cure, palliate, mitigate, or affect the diseases or the symptoms of the diseases. In specific embodiments of the present disclosure, the disease is a tumor or cancer defined as below.

As used herein, the term “cancer” or “tumor” refers to abnormal cell growth and proliferation, whether malignant or benign, as well as all precancerous cells, cancer cells and tissues. In some embodiments, the tumor comprises lymphoma, hematoma, and solid tumor. Preferably, the tumor is one or more of adrenocortical carcinoma, uroepithelial carcinoma of the bladder, breast carcinoma, squamous cell carcinoma of the cervix, intracervical adenocarcinoma, cholangiocarcinoma, adenocarcinoma of the colon, lymphoid neoplasm, diffuse large B-cell lymphoma, esophageal carcinoma, pleomorphic glioblastoma, squamous cell carcinoma of the head and neck, smectochromatous cell carcinoma of the kidney, clear cell carcinoma of the kidney and papillary cell carcinoma of the kidney, acute myeloid leukemia, low-grade glioma of the brain, hepatocellular carcinoma, adenocarcinoma of the lung, squamous cell carcinoma of the lung, mesothelial cell carcinoma, ovarian carcinoma, pancreatic carcinoma, pheochromocytoma and paraganglioma, prostate carcinoma, rectal carcinoma, malignant sarcoma, melanoma, gastric carcinoma, testicular germ-cell tumors, thyroid cancer, thymus gland carcinoma, endometrial carcinoma, uterine sarcoma, uveal melanoma, multiple myeloma, acute gonorrheal leukemia Multiple myeloma, acute lymphoid leukemia, chronic lymphoid leukemia, chronic myelogenous leukemia, T-cell lymphoma and B-cell lymphoma; more preferably, the cancer is selected from one or more of myeloma, leukemia, and pancreatic cancer.

The term “neurological disease” as used herein refers to any disorder of the brain, spinal cord, or other parts of the nervous system that results in identifiable structural damage. In some embodiments, the neurological disease may be selected from one or more of Parkinson's disease, Huntington's disease, multiple system atrophy, motor neuron disease, Alzheimer's disease, traumatic brain injury, ischemic stroke, spinal cord disease, amyotrophic lateral sclerosis, multiple sclerosis, and seizures.

In some embodiments, the autoimmune disease may be selected from one or more of systemic lupus erythematosus, rheumatoid arthritis, systemic sclerosis, Sjogren's syndrome, and polymyositis.

In some embodiments, the mTOR inhibitor refers to an allosteric inhibitor or a catalytic inhibitor that suppresses the mTOR signaling pathway; preferably, the mTOR inhibitor comprises one or more of Rapamycin (Sirolimus), Everolimus, Temsirolimus, Ridaforolimus (Deforolimus, MK8669), Sapanisertib (MLN0128), Vistusertib (AZD2014), and CC-115.

In some embodiments, the targeted protein degrader and its derivative therapeutics comprise at least one of a MGD and a PROTAC; preferably, the targeted protein degrader and its derivative therapeutics are MGDs; more preferably, the targeted protein degrader and its derivative therapeutics are immunomodulatory drugs.

In some embodiments, the MGD may comprise one or more of thalidomide derivatives, CC-90009, CC-885, sulfonamides, and CC-92480; preferably, the thalidomide derivatives comprise at least one of thalidomide, lenalidomide, pomalidomide, CC-220, and CC-122.

In some embodiments, the PROTAC may comprise one or more of ZNL-02096, ARV-771, ARV-110, ARV-471, KT-474, and NX-2127.

The terms “comprise” and “include” as used herein should be understood as inclusive rather than exclusive or exhaustive, meaning “including but not limited to.”

The term “therapeutically effective amount” generally refers to an amount sufficient to achieve a therapeutic effect for the listed diseases over an appropriate administration period.

The term “therapeutic” as used herein should be understood in its broadest sense, and does not necessarily imply complete recovery of the mammals. It includes alleviating symptoms, reducing the severity of an existing condition, or decreasing the frequency of acute episodes.

Unless otherwise defined, all technical and scientific terms used herein have the same meaning as commonly understood by a person skilled in the field. Any methods and materials similar or equivalent to those described herein can be used in the present disclosure. The preferred methods and materials described are for illustrative purposes only.

Before further describing specific embodiments of the present disclosure, it should be understood that the protection scope of the present disclosure is not limited to the particular specific embodiments described below. It should also be understood that the terms used in the embodiments of the present disclosure are intended to describe the particular embodiments and are not intended to limit the protection scope of the present disclosure. In the specification and claims of the present disclosure, unless the text expressly states otherwise, the singular forms “a”, “an” and “the” further include the plural form.

When an example provides a numerical range, it should be understood that, unless otherwise specified in the present disclosure, both endpoints of the range and any value between them can be selected. Unless otherwise defined, all technical and scientific terms used in the present disclosure have the same meaning as commonly understood by those skilled in the art. In addition to the specific methods, apparatuses, and materials used in the embodiments, any methods, apparatuses, and materials of the prior art that are similar or equivalent to the methods, apparatuses, and materials described in the embodiments of the present disclosure may be used to realize the present disclosure according to the knowledge of those skilled in the art and the disclosure of the present disclosure.

Unless otherwise indicated, the experimental methods, detection methods, and preparation methods disclosed in the present disclosure are conventional in the fields of molecular biology, biochemistry, chromatin structure and analysis, analytical chemistry, cell culture, recombinant DNA technology, and other related fields in the art.

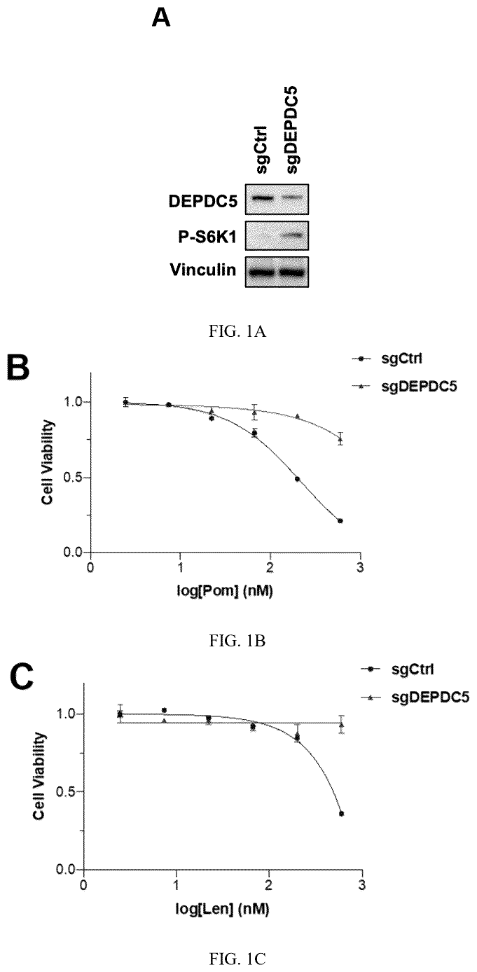

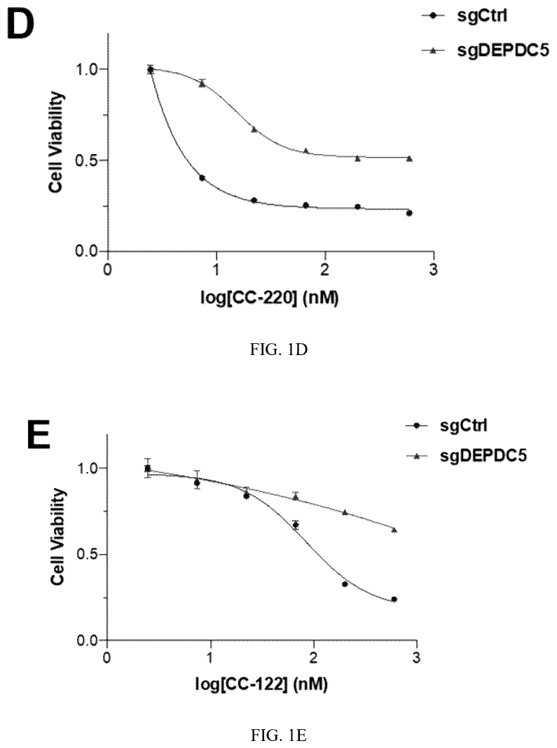

The present disclosure demonstrates that by knocking out the DEPDC5 gene to activate the mTOR signaling pathway (indicated by the phosphorylation activation of downstream S6K1), myeloma cells OPM2 acquire resistance to molecular glue degrader and its derivative therapeutics such as immunomodulatory drugs (IMiDs) (see FIG. 1). Additionally, the present disclosure further indicates that mTOR inhibitors, such as rapamycin, significantly enhance the cytotoxicity of pomalidomide against various myeloma cell lines (MM1S, NCI-H929, OPM2, U266, and RPMI-8226) with different sensitivities to IMiDs (see FIG. 1).

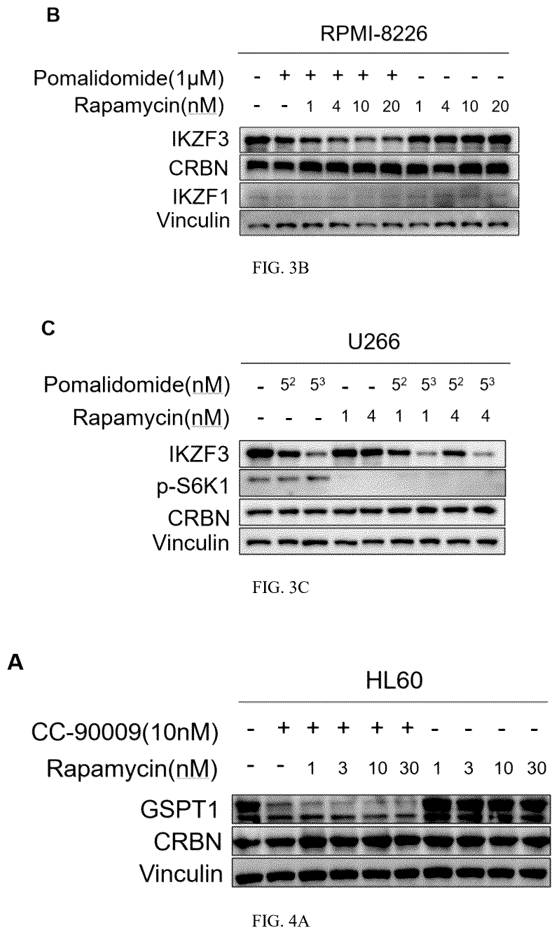

The present disclosure further investigates the molecular mechanism by which mTOR inhibitors increase myeloma's sensitivity to IMiDs. Experiments are conducted to explore whether mTOR inhibitors can enhance the degradation of substrate proteins by IMiDs. The experimental results show that the combination of rapamycin with IMiDs significantly improves the degradation of the substrate IKZF, which is crucial for the survival of myeloma cells, in myeloma cells OPM2, RPMI-8226, and U266 (see FIG. 3).

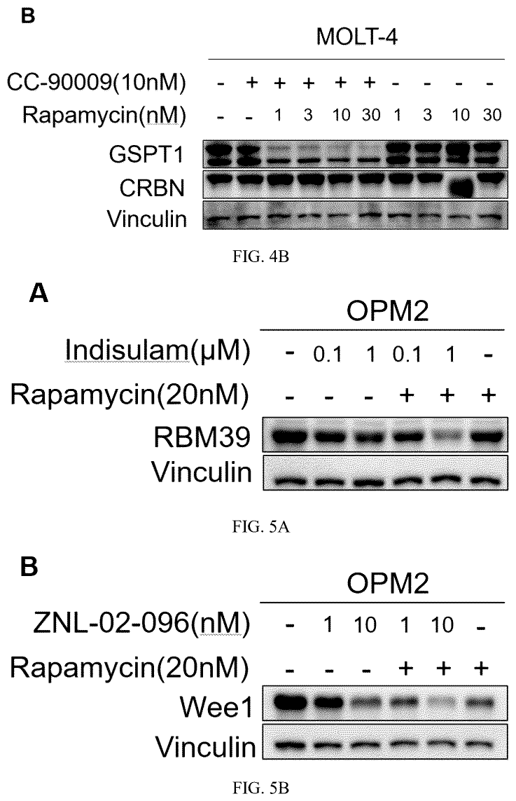

The scope of targeted protein degrader and its derivative therapeutics and their applicable diseases are then expanded to explore whether mTOR inhibitors can broadly enhance the efficacy of PROTAC and molecular glue protein degrader and its derivative therapeutics, developed from IMiDs, in various diseases. In human leukemia granulocyte cell line HL-60, human acute lymphoblastic leukemia cell line MOLT-4, and human pancreatic cancer cell line KP4, the combination of rapamycin with MGDs CC-90009 and CC-885 shows that rapamycin can still assist these targeted protein degraders and its derivative therapeutics to more effectively degrade the substrate GSPT1 (see FIG. 4). Subsequently, the combination effects of rapamycin with a MGD (indisulam), a PROTAC (ZNL-02-096 (CRBN-based) and a PROTAC ARV771 (VHL-based)) respectively are tested, showing enhanced degradation of substrates (see FIG. 5).

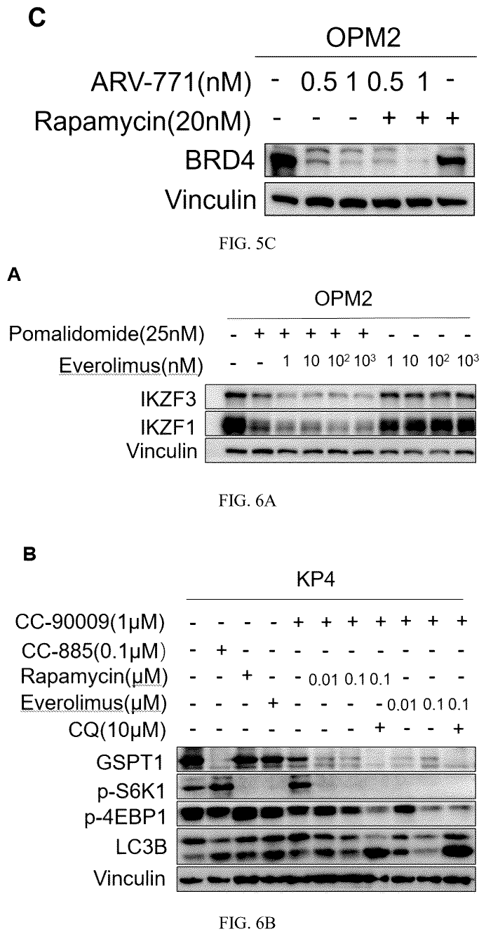

Additionally, the new-generation mTOR inhibitor everolimus is tested for its combined effects with IMiDs and/or other MGDs. Results show that everolimus can also enhance the degradation of substrates IKZFs and GSPT1, which is essential for cell survival (see FIG. 6).

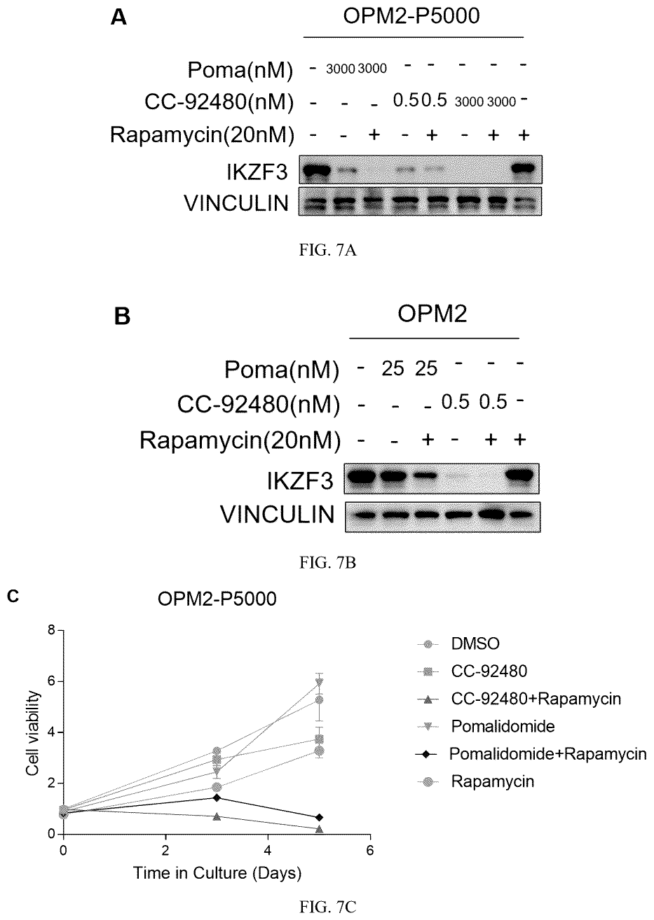

Finally, the combination effect of rapamycin with the new-generation IKZF3 degrader CC-92480 is tested. It has been found that rapamycin can enhance CC-92480's ability to degrade substrate proteins and improve its cytotoxic effect on myeloma cells (see FIG. 7).

### Embodiment 1 Resistance of DEPDC5 Knockout Myeloma Cell Line OPM2 to Immunomodulatory Drugs

### 1. Construction of DEPDC5 Knockout Myeloma Cell Line in OPM2

Construction of sgDEPDC5 Plasmid and Virus Packaging:

sgRNA targeting the DEPDC5 gene has a sequence shown as follows:

Forward:
(SEQ ID NO: 1)
caccgGCTACATCAGTGAAGATACC;
Reverse:
(SEQ ID NO: 2)
aaacGGTATCTTCACTGATGTAGCc

The above sgRNA was synthesized, annealed, diluted 100-fold, and then ligated to the lentiCRISPR-V2 plasmid. After transformation and picking single clones to check the successful and error-free ligation, plasmid extraction was performed. Viruses were packaged in 15 cm dishes. Plasmids (15 ug) were mixed with lentiviral packaging vectors pVSV-G (7.5 ug) and psPAX2 (11.25 ug), and co-incubated with the cell transfection reagent (Shanghai Life iLab Biotech Co., LTD) for 20 minutes, wherein a ratio of the cell transfection reagent to the plasmid mixture is 3:1. The incubated mixture was evenly added to 293FT cells in the logarithmic growth phase, followed by medium refreshment the next day. The supernatant was collected on the fourth day, filtered through a 0.45 μM filter, and concentrated using lenti-X-concentrator virus concentration solution (Clontech) overnight, yielding 1 ml/dish of concentrated virus solution.

### Lentiviral Infection on Suspended Myeloma Cell Line OPM2:

OPM2 cells were seeded in a 12-well plate at a density of 2 M/well. 300-400 μl of concentrated virus solution was added, along with polybrene at a final concentration of 10 μg/ml. The plate was centrifuged at 36° C., 800 g for 120 minutes. After centrifugation, the liquid of the 12-well plate was transferred to a T_{12.5} flask. The medium was refreshed the next day. Puromycin selection started on the third day to test for successful transduction. Cells in the control group were almost completely killed by puromycin due to the lack of resistance, while live cells in the experimental group indicated successful transduction.

FIG. 1A shows the efficiency of construction of DEPDC5-KO cell lines in the myeloma cell line OPM2 using Western blot analysis. The protein level of DEPDC5 in the constructed DEPDC5 knockout cell line is partially reduced compared to the control group. The expression level of p-S6K1, a downstream marker of the activated mTOR signaling pathway, is elevated, indicating activation of the mTOR signaling pathway in the DEPDC5 knockout cell line and confirming successful knockout of DEPDC5.

### 2. Cell Viability Detection Under Treatment of Immunomodulatory Drugs and Other MGDs

(1) OPM2 cells in the logarithmic growth phase were seeded in a 96-well plate at 3000 cells/well. The volume of the cell suspension is 90 μl/well.
(2) Each well was added with 10 μl lenalidomide (Len), pomalidomide (Pom), CC-122, or CC-220 down the concentration gradients starting from 1 μM. Control groups with no drug treatment were set. Each drug had 7 gradients with 3 replicates.
(3) The plate was placed in a humidity-controlled environment (37° C., 5% CO₂) for 5 days.
(4) Subsequently, 50 μl of CTL solution was added to each well under dark conditions. Then, the plate was placed on a shaker and shaken for 10 minutes, followed by reading on a microplate reader.
(5) The average absorbance values of the 3 replicates were used to plot the cell viability curves of OPM2 under drug treatment.

Cell viability was measured using the CellTiter-Lumi luminescent cell viability assay kit (Beyotime), with luminescence intensity reflecting cell viability, through an ATP-catalyzed luciferase reaction in the cell culture medium.

### Results:

Activation of the mTOR signaling pathway enables myeloma cells OPM2 to acquire resistance to immunomodulatory drugs. DEPDC5 is part of the GATOR1 complex, and the GATOR-Rag GTPase signaling pathway can inhibit mTORC1 activation. FIGS. 1B˜E show that knockout of DEPDC5 can activate the mTOR signaling pathway. After generation of the DEPDC5-KO cell lines in myeloma cell line OPM2, the cells were treated with immunomodulatory drugs lenalidomide (Len), pomalidomide (Pom), and next-generation MGDs CC-122 and CC-220. It can be observed that, compared to the control group, the DEPDC5 knockout myeloma cells exhibit resistance to MGDs such as those of immunomodulatory drugs.

### Embodiment 2 Killing Effect of Immunomodulatory Drug Pomalidomide in Combination with Rapamycin on Myeloma Cells

In this embodiment, the pomalidomide-sensitive myeloma cell lines (MM1S, H929 and OPM2) and the pomalidomide-resistant myeloma cell lines (U266 and RPMI-8226) were treated with mTOR signaling pathway inhibitor rapamycin alone, pomalidomide alone, a combination of rapamycin and pomalidomide, and control, respectively. Cell viability was assessed on Oh, 48 h, 96 h and 120 h post drug treatment. The detection used on each day is the same and has been described in Embodiment 1.

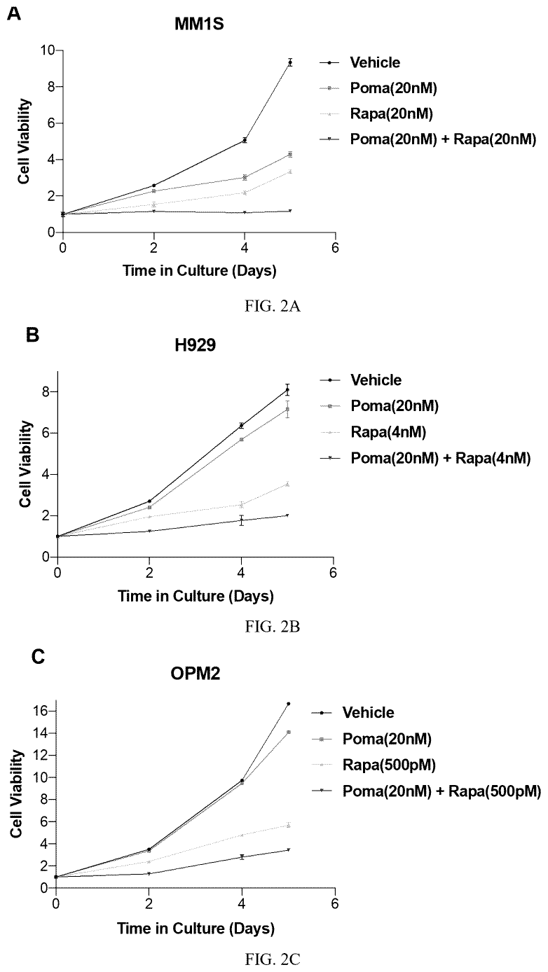

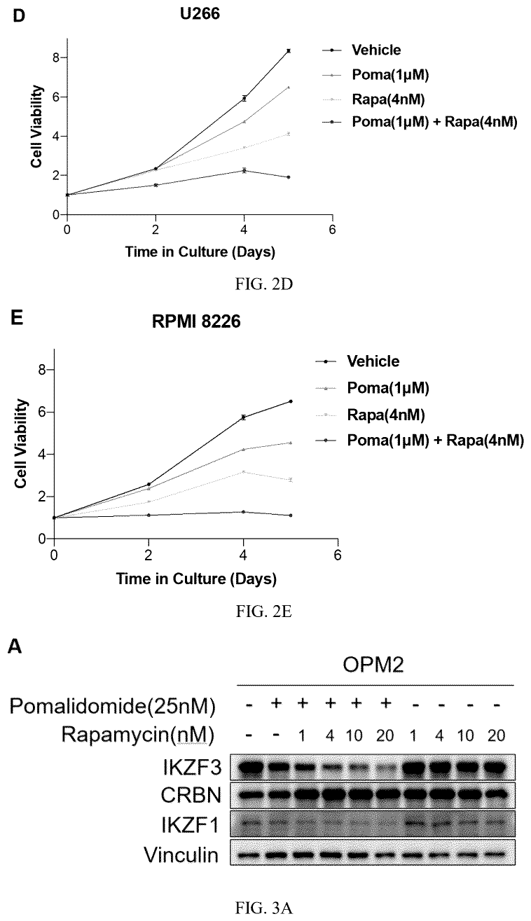

The results in FIG. 2 show that, for these five cell lines, the combination of pomalidomide (Pom) with the mTOR signaling pathway inhibitor rapamycin (RAPA) has a stronger effect in killing myeloma cells than pomalidomide alone. The data in FIG. 2 represent results from two independent experiments (n=3, mean±s.e.m., P<0.001).

### Embodiment 3 Enhanced Degradation of IKZF3 and IKZF1 by Pomalidomide in Combination with Rapamycin

25 nM of pomalidomide and varying concentrations of rapamycin were added to myeloma cell lines OPM2, RPMI-8226, and U266, respectively. Cell pellets were collected after 24 h and were lysed using RIPA. The protein levels were analyzed using Western blot.

As shown in FIG. 3, the level of the substrate IKZF3 remains unchanged when rapamycin is administered alone; whereas the level of IKZF3 is significantly reduced when pomalidomide and rapamycin are co-administered, indicating that co-administration enhances the degradation of IKZF3 and IKZF1 by pomalidomide.

### Embodiment 4 Enhanced Degradation of GSPT1 by CC90009 or CC-885 in Combination with Rapamycin

10 nM of CC90009 and varying concentrations of rapamycin were added to leukemia cell lines HL60 and MOLT-4, respectively. The samples were collected after 12 h, and the protein levels were examined by the same method as in Embodiment 3.

As shown in FIG. 4, the level of substrate protein GSPT1 of CC90009 remains unchanged when rapamycin is administered alone; whereas the level of GSPT1 is significantly reduced when CC90009 or CC-885 are co-administered with rapamycin, indicating that co-administration enhances the degradation of GSPT1 by CC90009 or CC-885.

### Embodiment 5 Enhanced Degradation of Substrate by Sulfonamides or PROTACs in Combination with Rapamycin

Different concentrations of indisulam (0, 0.1, 1 μm) and a fixed concentration of rapamycin (20 nM) were added to the myeloma cell line OPM2. After 24 hours, protein levels were analyzed using the same method as in Embodiment 3. FIG. 5a shows that at a working concentration of indisulam (1 μM), the combination with rapamycin allows indisulam to have a stronger degradation effect on the substrate RBM39 of indisulam (Vinculin as the internal reference).

Different concentrations of ZNL-02-096 (0, 1, 10 nM) and a fixed concentration of rapamycin (20 nM) were added to the myeloma cell line OPM2. After 24 hours, protein levels were detected using the same method as in Embodiment 3. FIG. 5b shows that at a working concentration of ZNL-02-096 (10 nM), the combination with rapamycin results in a stronger degradation effect on the substrate Wee1 of ZNL-02-096 (Vinculin as the internal reference).

Different concentrations of ARV-771 (0, 0.5, 1 nM) and a fixed concentration of rapamycin (20 nM) were added to the myeloma cell line OPM2. After 24 hours, protein levels were detected using the same method as in Embodiment 3. FIG. 5c shows that at working concentrations of ARV-771 (0.5 nM or 1 nM), the combination with rapamycin results in a stronger degradation effect on the substrate BRD4 of ARV-771 (Vinculin as the internal reference). The above results indicate that co-administration of rapamycin enhances the degradation of substrates by the sulfonamides or PROTACs.

The indisulam used in this embodiment is a type of sulfonamide, also a MGD. Indisulam can direct Dcaf15 to degrade RBM39. ZNL-02-096 is a PROTAC, and its substrate is Wee1. It was first synthesized at the Dana-Farber Cancer Institute in 2020(Li et al (2020) Development and characterization of a Wee1 kinase degrader. Cell Chem. Biol. 27 57 PMID: 31735695). ARV771 is also a PROTAC that can direct VHL to degrade BET proteins, such as BRD4 (Raina et al (2016) PROTAC-induced BET protein degradation as a therapy for castration-resistant prostate cancer. Proc Natl Acad Sci USA. 113 7124 PMID: 27274052).

### Embodiment 6 Enhanced Degradation of IKZF3 and IKZF1 by Pomalidomide in Combination with the mTOR Signaling Pathway Inhibitor Everolimus

In addition to rapamycin, the mTOR signaling pathway inhibitor includes everolimus. Everolimus, as a rapamycin derivative, has been approved by the U.S. Food and Drug Administration (FDA) for clinical use.

Different concentrations of ZNL-02-096 (0, 1, 10, 10², 10³ nM) and a fixed concentration of pomalidomide (25 nM) were added to the myeloma cell line OPM2. After 24 hours, protein levels were detected using the same method as in Embodiment 3. FIG. 6a shows that the combinations with varying concentrations of everolimus allow pomalidomide to have a stronger degradation effect on the substrates IKZF3 and IKZF1 in the myeloma cell line OPM2.

Different concentrations of rapamycin or everolimus (0, 0.01, 0.1 μM), as well as a fixed concentration of CC-90009 (1 μM) or CC-885 (0.1 μM) were added to the human pancreatic cancer cell line KP4. After 24 hours, protein levels were detected using the same method as in Embodiment 3. FIG. 6b shows that the combination with varying concentrations of rapamycin or everolimus allows CC-90009 or CC-885 to have a stronger degradation effect on the substrate GSPT1 in KP4.

### Embodiment 7 Enhanced Degradation of IKZF3 and Increased Killing Effect on Myeloma Cells by CC-92480 in Combination with Rapamycin

In this embodiment, the myeloma cell line OPM2, which is sensitive to pomalidomide was subjected to long-term high-dose pomalidomide treatment to obtain the pomalidomide-resistant cell line OPM2-P5000. Pomalidomide and CC-92480 (0.5 nM, 25 nM) were administered to OPM2 cells, and pomalidomide and CC-92480 (0.5 nM, 3 μM) were administered to OPM2-P5000 cells. These treatments were combined with a fixed concentration of rapamycin (20 nM).

FIG. 7a and FIG. 7b show that in the pomalidomide-resistant myeloma cell line, co-administration with rapamycin is able to enhance the degradation of the substrate IKZF3 by pomalidomide or CC-92480.

Meanwhile, this embodiment provides the following groups for cell viability test in the pomalidomide-resistant myeloma cell line OPM2-P5000: rapamycin alone (20 nM), pomalidomide alone (3 μM), CC-92480 alone (3 μM), combination treatment, and control. Cell viability was assessed on Oh, 96 h and 120 h post drug treatment using the same method as in Embodiment 1.

FIG. 7c shows that CC-92480 has a stronger killing effect on myeloma cells when co-administered with rapamycin (n=3, mean±s.e.m.).

In summary, the present disclosure provides a method of enhancing the efficacy of a targeted protein degrader and its derivative therapeutics, expanding the application of mTOR inhibitors and solving the problems of drug resistance and recurrence of a variety of diseases, such as tumors. Therefore, the present disclosure effectively overcomes the drawbacks of the prior art and has high industrial value.

The above embodiments are intended to illustrate the embodiments of the present disclosure and should not be construed as a limitation of the present disclosure. Furthermore, the various modifications set forth herein, as well as variations in methods and compositions of the present disclosure, will be apparent to those skilled in the art without departing from the scope and spirit of the present disclosure. Although the present disclosure has been specifically described in connection with a variety of specific preferred embodiments of the present disclosure, it should be understood that the present disclosure is not limited thereto. Additionally, various modifications to the present disclosure as described above that would be obvious to one skilled in the art should be included within the scope of the present disclosure.

What is claimed is:

1. Use of mTOR inhibitors in the preparation of products for enhancing the efficacy of targeted protein degrader and its derivative therapeutics.

2. The use of claim 1, wherein the mTOR inhibitors refer to allosteric inhibitors or catalytic inhibitors capable of inhibiting the mTOR signaling pathway.

3. The use of claim 1, wherein the mTOR inhibitors comprise one or more of Rapamycin (Sirolimus), Everolimus, Temsirolimus, Ridaforolimus (deforolimus, MK-8669), Sapanisertib (MLN0128), Vistusertib (AZD2014), and CC-115.

4. The use of claim 1, wherein the targeted protein degrader and its derivative therapeutics comprises at least one of a molecular glue degrader (MGD) and a Proteolysis Targeting Chimera (PROTAC).

5. The use of claim 1, wherein the targeted protein degrader is an immunomodulatory drug.

6. The use of claim 4, wherein
1) the molecular glue degraders comprise one or more of thalidomide, thalidomide derivatives, CC-90009, CC-885, sulfonamides and CC-92480;
2) the PROTACs comprise one or more of ZNL-02-096, ARV-771, ARV-110, ARV-471, KT-474 and NX-2127.

7. The use of claim 6, wherein the thalidomide derivatives comprise at least one of lenalidomide, pomalidomide, CC-220 and CC-122.

8. The use of claim 1, wherein enhancing the efficacy of a targeted protein degrader comprises at least any one of the following:
1) enhancing the ability of the targeted protein degrader to degrade a substrate;
2) enhancing the sensitivity of a substrate to the targeted protein degrader;
3) reducing the resistance to the targeted protein degrader;
4) enhancing the inhibitory effect of the targeted protein degrader on tumor cell viability;
5) enhancing the therapeutic effect of the targeted protein degrader on disease progression.

9. The use of claim 8, comprising at least any one of the following:
1) enhancing the killing effect of pomalidomide on myeloma cells;
2) enhancing the degradation of substrates by pomalidomide;
3) enhancing the degradation of substrates by CC-90009 or CC-885;
4) enhancing the degradation of substrates by indisulam;
5) enhancing the degradation of substrates by the PROTACs;
6) enhancing the degradation of substrates by CC-92480;
7) enhancing the killing effect of CC-92480 on myeloma cells.

10. The use of claim 9, comprising at least any one of the following:
1) the myeloma cells are sensitive to pomalidomide or resistant to pomalidomide;
2) enhancing the degradation of substrates IKZF3 and IKZF1 by pomalidomide;
3) enhancing the degradation of a substrate GSPT1 by CC-90009 or CC-885;
4) enhancing the degradation of a substrate RBM39 by indisulam;
5) enhancing the degradation of a substrate Wee1 by ZNL-02-096 or ARV-771;
6) enhancing the degradation of a substrate IKZF3 by CC-92480;
7) enhancing the killing effect of CC-92480 on pomalidomide-sensitive or pomalidomide-resistant myeloma cells.

11. The use of claim 9, comprising enhancing the killing effect of CC-92480 on a pomalidomide-sensitive myeloma cell line OPM2 and a pomalidomide-resistant myeloma cell line OPM2-P5000.

12. The use of claim 1, wherein the targeted protein degrader is used to treat diseases selected from one or more of tumors, neurological diseases, autoimmune diseases, infectious diseases and inflammatory diseases.

13. The use of claim 12, wherein the tumors are selected from one or more of lymphoma, hematologic malignancies and solid tumors;
and/or, the neurological diseases are selected from one or more of Parkinson's disease, Huntington's disease, multiple system atrophy, motor neuron diseases, Alzheimer's disease, traumatic brain injury, ischemic stroke, spinal cord diseases, amyotrophic lateral sclerosis, multiple sclerosis and epilepsy;
and/or, the autoimmune diseases are selected from one or more of systemic lupus erythematosus, rheumatoid arthritis, systemic sclerosis, Sjogren's syndrome and polymyositis.

14. The use of claim 12, wherein the tumor is selected from one or more of adrenocortical carcinoma, uroepithelial carcinoma of the bladder, breast carcinoma, squamous cell carcinoma of the cervix, intracervical adenocarcinoma, cholangiocarcinoma, adenocarcinoma of the colon, lymphoid neoplasm, diffuse large B-cell lymphoma, esophageal carcinoma, pleomorphic glioblastoma, squamous cell carcinoma of the head and neck, smectochromatous cell carcinoma of the kidney, clear cell carcinoma of the kidney and papillary cell carcinoma of the kidney, acute myeloid leukemia, low-grade glioma of the brain, hepatocellular carcinoma, adenocarcinoma of the lung, squamous cell carcinoma of the lung, mesothelial cell carcinoma, ovarian carcinoma, pancreatic carcinoma, pheochromocytoma and paraganglioma, prostate carcinoma, rectal carcinoma, malignant sarcoma, melanoma, gastric carcinoma, testicular germ-cell tumors, thyroid cancer, thymus gland carcinoma, endometrial carcinoma, uterine sarcoma, uveal melanoma, multiple myeloma, acute gonorrheal leukemia Multiple myeloma, acute lymphoid leukemia, chronic lymphoid leukemia, chronic myelogenous leukemia, T-cell lymphoma and B-cell lymphoma.

15. The use of claim 12, wherein the tumor is one or more of myeloma, leukemia and pancreatic cancer.

16. A pharmaceutical composition, comprising a targeted protein degrader and a mTOR inhibitor, as well as pharmaceutically acceptable excipients.

17. The pharmaceutical composition of claim 16, wherein the mTOR inhibitors refer to allosteric inhibitors or catalytic inhibitors capable of inhibiting the mTOR signaling pathway.

18. The pharmaceutical composition of claim 16, wherein the mTOR inhibitors include one or more of Rapamycin (Sirolimus), Everolimus, Temsirolimus, Ridaforolimus (deforolimus, MK-8669), Sapanisertib (MLN0128), Vistusertib (AZD2014), and CC-115.

19. The pharmaceutical composition of claim 16, wherein the targeted protein degrader and its derivative therapeutics comprises at least one of a molecular glue degrader (MGD) and a Proteolysis Targeting Chimera (PROTAC).

20. The pharmaceutical composition of claim 16, wherein the targeted protein degrader is an immunomodulatory drug.

21. The pharmaceutical composition of claim 16, wherein the targeted protein degrader is used to treat diseases selected from one or more of tumors, neurological diseases, autoimmune diseases, infectious diseases and inflammatory diseases.

22. The pharmaceutical composition of claim 21, wherein the tumors are selected from one or more of lymphoma, hematologic malignancies and solid tumors;
and/or, the neurological diseases are selected from one or more of Parkinson's disease, Huntington's disease, multiple system atrophy, motor neuron diseases, Alzheimer's disease, traumatic brain injury, ischemic stroke, spinal cord diseases, amyotrophic lateral sclerosis, multiple sclerosis and epilepsy;
and/or, the autoimmune diseases are selected from one or more of systemic lupus erythematosus, rheumatoid arthritis, systemic sclerosis, Sjogren's syndrome and polymyositis.

23. The pharmaceutical composition of claim 21, wherein the tumor is selected from one or more of adrenocortical carcinoma, uroepithelial carcinoma of the bladder, breast carcinoma, squamous cell carcinoma of the cervix, intracervical adenocarcinoma, cholangiocarcinoma, adenocarcinoma of the colon, lymphoid neoplasm, diffuse large B-cell lymphoma, esophageal carcinoma, pleomorphic glioblastoma, squamous cell carcinoma of the head and neck, smectochromatous cell carcinoma of the kidney, clear cell carcinoma of the kidney and papillary cell carcinoma of the kidney, acute myeloid leukemia, low-grade glioma of the brain, hepatocellular carcinoma, adenocarcinoma of the lung, squamous cell carcinoma of the lung, mesothelial cell carcinoma, ovarian carcinoma, pancreatic carcinoma, pheochromocytoma and paraganglioma, prostate carcinoma, rectal carcinoma, malignant sarcoma, melanoma, gastric carcinoma, testicular germ-cell tumors, thyroid cancer, thymus gland carcinoma, endometrial carcinoma, uterine sarcoma, uveal melanoma, multiple myeloma, acute gonorrheal leukemia Multiple myeloma, acute lymphoid leukemia, chronic lymphoid leukemia, chronic myelogenous leukemia, T-cell lymphoma and B-cell lymphoma.

24. The pharmaceutical composition of claim 21, wherein the tumor is one or more of myeloma, leukemia and pancreatic cancer.

25. A method for treating diseases, comprising administering to an individual or subject in need thereof a therapeutically effective amount of a targeted protein degrader and a mTOR inhibitor, or the pharmaceutical composition of any one of claims 16-24; wherein the diseases are selected from one or more of tumors, neurological diseases, autoimmune diseases, infectious diseases and inflammatory diseases.

26. The method of claim 25, wherein the tumors are selected from lymphoma, hematologic malignancies and solid tumors;
and/or, the neurological diseases are selected from one or more of Parkinson's disease, Huntington's disease, multiple system atrophy, motor neuron diseases, Alzheimer's disease, traumatic brain injury, ischemic stroke, spinal cord diseases, amyotrophic lateral sclerosis, multiple sclerosis and epilepsy;
and/or, the autoimmune diseases are selected from one or more of systemic lupus erythematosus, rheumatoid arthritis, systemic sclerosis, Sjogren's syndrome and polymyositis.

27. The method of claim 25, wherein the tumor is selected from one or more of adrenocortical carcinoma, uroepithelial carcinoma of the bladder, breast carcinoma, squamous cell carcinoma of the cervix, intracervical adenocarcinoma, cholangiocarcinoma, adenocarcinoma of the colon, lymphoid neoplasm, diffuse large B-cell lymphoma, esophageal carcinoma, pleomorphic glioblastoma, squamous cell carcinoma of the head and neck, smectochromatous cell carcinoma of the kidney, clear cell carcinoma of the kidney and papillary cell carcinoma of the kidney, acute myeloid leukemia, low-grade glioma of the brain, hepatocellular carcinoma, adenocarcinoma of the lung, squamous cell carcinoma of the lung, mesothelial cell carcinoma, ovarian carcinoma, pancreatic carcinoma, pheochromocytoma and paraganglioma, prostate carcinoma, rectal carcinoma, malignant sarcoma, melanoma, gastric carcinoma, testicular germ-cell tumors, thyroid cancer, thymus gland carcinoma, endometrial carcinoma, uterine sarcoma, uveal melanoma, multiple myeloma, acute gonorrheal leukemia Multiple myeloma, acute lymphoid leukemia, chronic lymphoid leukemia, chronic myelogenous leukemia, T-cell lymphoma and B-cell lymphoma.

28. The method of claim 25, wherein the tumor is one or more of myeloma, leukemia and pancreatic cancer.

29. The method of claim 25, wherein the mTOR inhibitors refer to allosteric inhibitors or catalytic inhibitors capable of inhibiting the mTOR signaling pathway;
and/or the targeted protein degrader and its derivative therapeutics comprises at least one of a molecular glue degrader (MGD) and a Proteolysis Targeting Chimera (PROTAC).

30. The method of claim 25, wherein the mTOR inhibitors include one or more of Rapamycin (Sirolimus), Everolimus, Temsirolimus, Ridaforolimus (deforolimus, MK-8669), Sapanisertib (MLN0128), Vistusertib (AZD2014) and CC-115;
and/or the targeted protein degrader and its derivative therapeutics is one or more of a molecular glue degrader (MGD) and a Proteolysis Targeting Chimera (PROTAC).

31. The method of claim 25, wherein the targeted protein degrader is an immunomodulatory drug.

32. The method of claim 29, wherein the molecular glue degrader comprises one or more of thalidomide, thalidomide derivatives, CC-90009, CC-885, sulfonamides and CC-92480;
the PROTACs comprise one or more of ZNL-02-096, ARV-771, ARV-110, ARV-471, KT-474 and NX-2127.

33. The use of claim 32, wherein the thalidomide derivatives comprise at least one of lenalidomide, pomalidomide, CC-122 and CC-220.
